(19) United States
(12) Patent Application Publication
(10) Pub. No.: US 2020/0138631 A1
(43) Pub. Date: May 7, 2020

(54) SPORTS MUSCLE CARE TAPE

(71) Applicant: Jong Suk PARK, Incheon (KR)

(72) Inventor: Jong Suk PARK, Incheon (KR)

(21) Appl. No.: 16/502,879

(22) Filed: Jul. 3, 2019

(30) Foreign Application Priority Data
Nov. 6, 2018 (KR) 10-2018-0134885

(51) Int. Cl. A61F 13/02; C09J 7/20

(52) U.S. Cl. CPC A61F 13/02; C09J 7/30; C09J 7/20

(57) ABSTRACT

A sports muscle care tape includes: a substrate tape extending in a lengthwise direction; and an adhesive layer formed on a bottom surface of the substrate tape. The adhesive composition comprises germanium, a polymer represented by formula 1, sorbic acid, a cucumber extract, a pineapple extract, a Dendropanax morbifera Lev extract, 1,3-butylene glycol, tourmaline, zeolite, a photocatalyst, and activated carbon. The sports muscle care tape may perform the basic functions of a sports tape and, at the same time, effectively protect an affected part by absorbing external impact. In addition, the sports muscle care tape may promote blood circulation and metabolism, has antibacterial activity, may deodorize the sweat, odor and the like generated in taped skin, and thus can be hygienically used.

### BACKGROUND

The present invention relates to a sports muscle care tape, and more particularly to a sports muscle care tape which can perform the basic functions of a sports tape and, at the same time, effectively protect an affected part by absorbing external impact.

Nowadays, modern people suffer from pain caused by various factors. Specifically, various factors, such as exercise or work, improper posture, and mental stress, can put strain on the muscles, and thus cause pain. For example, long-term computer work may result in stiff shoulder muscles, and if a person walks or stands for a long time, the person may have various symptoms such as swelling of the leg muscles.

If the muscles are tensed or damaged as described above, the muscles can be swollen and the circulation of blood, lymphatic fluid or tissue fluid cannot become smooth, resulting in pain. In this case, when a tape having elasticity similar to the elasticity of the muscles is attached to the painful area, it can increase the space between the skin and the muscles, thus increasing the circulation of blood, lymphatic fluid or tissue fluid, resulting in relief of pain, and can help relieve pain by skin stimulation. Attaching this specially processed tape to help relieve pain is called taping therapy.

This taping therapy was developed in 1970s by Dr. Kase Kenzo (Japanese) during studies on sports taping for the purpose of treating myositis and arthritis. This therapy is a method of providing a preventive effect before exercise by attaching a tape having the same elasticity as that of human body muscles to a painful area or the skin to stretch the muscles of the corresponding part, and may have the effects of relieving pain and preventing injury.

According to the principle of this therapy, a tape having the same contractile force of human body muscles lifts the skin, and thus provides a space between the skin and the muscles, thereby increasing the circulation of blood, lymphatic fluid and neurotransmitters, resulting in improvement in the self-healing power of the painful area. The taping therapy is a non-surgical, non-pharmacologic therapy with little side effects, and can easily be applied to both men and women, including pregnant women, children, the elderly, and so on. The taping therapy is a method of attaching a tape to the muscle area of interest in order to normalize the action of moving (agonistic) muscles, and can reduce or increase the tension of the muscles and promote the circulation of blood, lymphatic fluid and tissue fluid. In particular, Kinesio taping is a technique that attaches an elastic functional tape depending on the texture of muscles that move joints, thereby increasing the circulation of blood and lymphatic fluid between the muscles and the skin, resulting in improvement in the motor function of the muscles, and is used for therapeutic purposes. This Kinesio taping technique is widely used not only in the sports field, but also in daily life, and can be applied without any particularly worrying side effects.

### SUMMARY OF THE INVENTION

An object of the present invention is to provide a sports muscle care tape which can perform the basic functions of a sports tape and, at the same time, effectively protect an affected part by absorbing external impact.

Another object of the present invention is to provide a sports muscle care tape which can promote blood circulation and metabolism, has antibacterial activity, can deodorize the sweat, odor and the like generated in taped skin, and thus can be hygienically used.

Various objects to be achieved by the present invention are not limited to the above-mentioned objects, and other objects which are mentioned herein can be clearly understood by those skilled in the art from the following description.

A sports muscle care tape according to the present invention includes: a substrate tape extending in a lengthwise direction; and an adhesive layer formed on the bottom surface of the substrate tape, wherein the adhesive layer includes 100 parts by weight of an adhesive and 5 to 15 parts by weight of an adhesive composition, wherein the adhesive composition includes germanium, a polymer represented by the following formula 1, sorbic acid, a cucumber extract, a pineapple extract, a Dendropanax morbifera Lev extract, 1,3-butylene glycol, tourmaline, zeolite, a photocatalyst, and activated carbon:

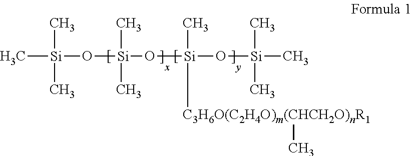

wherein x is an integer ranging from 500 to 30,000; y is an integer ranging from 100 to 10,000; m and n are each independently an integer ranging from 10 to 10,000; and R₁ is hydrogen or an alkyl group having 2 to 5 carbon atoms.

The adhesive composition may include 1 to 3 parts by weight of germanium, 5 to 15 parts by weight of the polymer represented by formula 1, 3 to 5 parts by weight of sorbic acid, 1 to 3 parts by weight of the cucumber extract, 0.5 to 1.5 parts by weight of the pineapple extract, 2 to 4 parts by weight of the Dendropanax morbifera Lev extract, 5 to 10 parts by weight of 1,3-butylene glycol, 1 to 3 parts by weight of tourmaline, 2 to 4 parts by weight of zeolite, 2 to 4 parts by weight of the photocatalyst, and 0.5 to 1.5 parts by weight of activated carbon, and the photocatalyst may be composed of a mixture of 1 to 3 parts of titanium dioxide and 0.1 to 0.5 parts by weight of silver nanopowder.

The details of other embodiments are included in the following detailed description.

### BRIEF DESCRIPTION OF THE DRAWINGS

FIG. 1 schematically illustrates a sports muscle care tape according to the present invention.

FIG. 2a is a photograph showing capillary blood flow before attachment of a sports muscle care tape prepared according to the present invention.

FIG. 2a is a photograph showing capillary blood flow after attachment of a sports muscle care tape prepared according to the present invention.

### DETAILED DESCRIPTION OF THE INVENTION

The advantages and features of the present invention, and the way of attaining them, will become apparent with reference to the exemplary embodiments described below in detail. However, the present invention is not limited to the exemplary embodiments disclosed below and can be embodied in a variety of different forms; rather, these embodiments are provided so that this disclosure will be thorough and complete, and will fully convey the scope of the present invention to those skilled in the art.

Terms used herein are only to describe specific embodiments and are not intended to limit the scope of the present invention. Singular expressions include plural expressions unless otherwise specified in the context thereof.

Unless otherwise defined, all teams used herein, including technical and scientific terms, have the same meaning as commonly understood by one of ordinary skill in the art to which the present invention belongs. The terms used in general and defined in dictionaries should be interpreted as having meanings identical to those specified in the context of related technology. Unless definitely defined in the present application, the terms should not be interpreted as having ideal or excessively formative meanings.

Hereinafter, preferred embodiments of a sports muscle care tape according to the present invention will be described in detail.

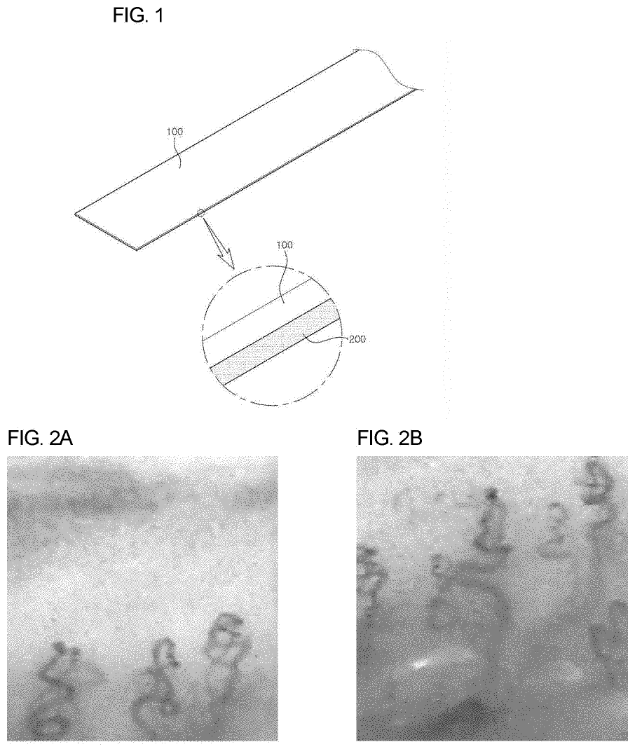

FIG. 1 schematically illustrates a sports muscle care tape according to the present invention.

Referring to FIG. 1, the sports muscle care tape according to the present invention includes: a substrate tape 100 extending in a lengthwise direction; and an adhesive layer 200 formed on the bottom layer of the substrate tape 100.

The substrate tape 100 is made of a fabric woven of elastic synthetic fiber. This substrate tape 100 has elasticity similar to those of the skin and muscles, and thus may function as a support that supports the movement of muscles and relaxes or contracts muscles.

The adhesive layer 200 is provided to attach the substrate tape 100 to the body, and the adhesive layer 200 may be formed by applying an adhesive including an adhesive composition to the bottom surface of the substrate tape 100.

The adhesive layer 200 may include 100 parts by weight of a conventional adhesive, which is used to manufacture tapes, and 5 to 15 parts by weight of an adhesive composition, and may constitute the sports muscle care tape.

In addition, the adhesive layer 200 may further include, in addition to 100 parts by weight of the conventional adhesive and the adhesive composition, glucosamine, methyl sulfonyl methane (dietary sulfur), and vanillyl butyl ether. The glucosamine, methyl sulfonyl methane and vanillyl butyl ether included in the adhesive layer 200 may promote heat generation, thereby promoting blood circulation and metabolism in the taped body part.

In the present invention, the above-described glucosamine, methyl sulfonyl methane and vanillyl butyl ether may be included in amounts of 1 to 5 parts by weight, 3 to 7 parts by weight and 5 to 10 parts by weight, respectively, based on 100 parts by weight of the adhesive composition.

The adhesive composition may include germanium, a polymer represented by formula 1, sorbic acid, a cucumber extract, a pineapple extract, a Dendropanax morbifera Lev extract, 1,3-butylene glycol, tourmaline, zeolite, a photocatalyst, and activated carbon.

In addition, in the present invention, the adhesive composition may include 1 to 3 parts by weight of germanium, 5 to 15 parts by weight of the polymer represented by formula 1, 3 to 5 parts by weight of sorbic acid, 1 to 3 parts by weight of the cucumber extract, 0.5 to 1.5 parts by weight of the pineapple extract, 2 to 4 parts by weight of the Dendropanax morbifera Lev extract, 5 to 10 parts by weight of 1,3-butylene glycol, 1 to 3 parts by weight of tourmaline, 2 to 4 parts by weight of zeolite, 2 to 4 parts by weight of the photocatalyst, and 0.5 to 1.5 parts by weight of activated carbon, and the photocatalyst may be composed of a mixture of 1 to 3 parts of titanium dioxide and 0.1 to 0.5 parts by weight of silver nanopowder.

Germanium which is used in the present invention is a rare element, and is a lustrous, gray-white semi-metallic element. It is a semiconductor having mysterious pharmacological effects, and is closest to “Ki” among the elements present on Earth. In addition, it is an element having strong “Ki”, and is known as an element of mystery, an element of life, or a miracle material.

This germanium may be classified into organic germanium (Ge132) and inorganic germanium (Ge32). Only organic germanium can be used in the human body. Organic germanium is edible oxygen and is known as hydrogen-rich antioxidant in modern science.

In addition, germanium can provide an ionized antioxidant which can activate cells, promote growth, and convert acidic water to alkaline water beneficial to the human body. In addition, it can emit far infrared rays that enhance physiological activity and metabolism.

Germanium is known to emit a large amount of far infrared rays. The far infrared rays have a wavelength of 4 to 1000 μm, and change into heat by penetrating deeply into the skin, and this The absorption action occurs mainly in the wavelength range of 5 to 30 μm. When a vibrational wavelength is absorbed into the human body, atoms and molecules can resonate in the living body and change into thermal energy which activates each cell, thus promoting metabolism.

Namely, germanium (Ge) is known to have excellent thermal resistance and low thermal expansion properties and emit a larger amount of far infrared rays than other oxides, and thus has been used as a far infrared ray-emitting material in many studies. When germanium comes into contact with the skin, germanium ions enter the body and resonate. When germanium is ingested, it is released together various toxic substances with from the body within 20 to 30 hours, indicating that it causes no addiction or side effects. In addition, it may perform functions, including deodorization, antifungal activity, dehumidification, air purification, and warming.

The polymer may be represented by the following formula 1:

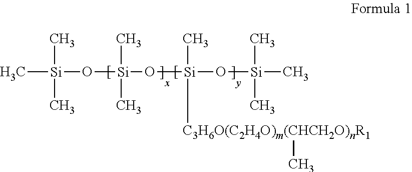

wherein x is an integer ranging from 500 to 30,000; y is an integer ranging from 100 to 10,000; m and n are each independently an integer ranging from 10 to 10,000; and R₁ is hydrogen or an alkyl group having 2 to 5 carbon atoms.

The polymer represented by formula 1 is a silicone glycol copolymer which has a polyethylene oxide group in the molecule and may also have an amino or epoxy group. The polymer represented by formula 1 can improve the flexibility and slippage of the muscle care tape and may be hydrophilic.

Sorbic acid which is used in the present invention is a typical synthetic preservative and is often used to preserve processed foods. Sorbic acid and benzoic acid and its salts are regarded as representative synthetic preservatives. This substance is highly effective in inhibiting the growth of various microorganisms, and dissolves well in glacial acetic acid and various organic solvents. When it is used in food, it may be mixed with acetic acid, lactic acid, alcohol, etc., or used in salt form such as potassium sorbate or calcium sorbate. The addition of sorbic acid can increase antibacterial activity, making it possible to have excellent antibacterial and deodorizing functions.

The cucumber extract that is used in the present invention may be prepared by the following method.

To prepare the cucumber extract, a cucumber fruit and a cucumber stem are harvested and washed with purified water containing 1 to 3 (w/w) % sodium hydrogen carbonate (NaHCO₃) dissolved therein at a temperature of 30 to 35° C. for 3 to 5 minutes to remove foreign matter attached to the cucumber fruit and stem.

Then, the washed cucumber fruit is dried in a dryer. Drying of the cucumber may be performed in a dryer which is maintained at a certain temperature and humidity are maintained in order to prevent the chlorophyll of the cucumber from being destroyed in sunlight drying and the characteristic scent of the cucumber from disappearing. For example, drying of the cucumber fruit may be performed in a dryer at a temperature of 20 to 25° C. and a humidity of 33 to 40% for 5 to 10 hours.

Thereafter, the dried cucumber fruit may be mixed with purified, and then heated, thereby preparing a cucumber fruit extract.

The cucumber fruit extract may be prepared by mixing 100 parts by weight of the dried cucumber fruits with 200 to 300 parts by weight of purified water, and then heating the mixture at a temperature of 95 to 105° C. for 5 to 10 hours.

Next, the cucumber stem is dried to remove water contained in the cucumber stem. Drying of the cucumber stem may be performed in a dryer at a temperature of 35 to 40° C. and a humidity of 35 to 45% for 10 to 15 hours. According to the present invention, when the cucumber stem is dried under the above-described conditions, water contained in the cucumber stem can be evaporated gradually and evenly, and thus the characteristic scent and useful components of the cucumber stem can be more easily exhibited. In addition, the cell structure of the cucumber stem can be destroyed, so that the penetration of air into the cucumber stem can be facilitated and the fermentation of the cucumber stem can be promoted, whereby the useful components of the cucumber stem can be extracted thickly.

Thereafter, the dried cucumber stem is mixed with purified water, and then heated, thereby preparing a cucumber stem extract. The cucumber stem extract may be prepared by mixing 100 parts by weight of the dried cucumber stem with 100 to 150 parts by weight of purified water, immersing the cucumber stem in the purified water at a temperature of 70 to 80° C. for 1 to 5 hours, and then introducing the purified water containing the cucumber stem immersed therein into a heater, and then heating the introduced purified water containing the cucumber stem immersed therein at a temperature of 95 to 105° C. and an autoclave internal pressure of 2.0 to 3.0 kgf/cm² for 3 to 5 hours.

According to the present invention, as described above, the cucumber stem is immersed in purified water, and then extracted. Thus, the efficiency of extraction of the cucumber stem can be increased by weakening the plant wall of the cucumber stem.

Thereafter, the cucumber fruit extract and the cucumber stem extract are mixed with each other, thereby preparing a cucumber extract. Specifically, the cucumber extract may be prepared by mixing the cucumber fruit extract and the cucumber stem extract at a weight ratio of 8:2 to 6:4.

Then, the cucumber extract may be aged by storing it at a temperature of 5 to 10° C. for 1 to 5 hours. When the cucumber extract is aged under these conditions, the cucumber extract can be mixed uniformly and the fragrance thereof can be enhanced.

The pineapple extract that is used in the present invention may be prepared by the following method.

First, pineapple is prepared, and then washed to remove foreign matter.

Washing of the pineapple may be performed using purified water containing sodium hydrogen carbonate (NaHCO₃) dissolved therein. When the surface of the pineapple is washed with the purified water containing sodium hydrogen carbonate dissolved therein, agricultural chemical components, heavy metals and fungi, which remain the surface of the pineapple, can be efficiently washed out.

The sodium hydrogen carbonate (NaHCO₃) is also used as a food additive, is not toxic, and can be used to wash the surface of the pineapple clean because it can penetrate, diffuse and expand. Preferably, the sodium hydrogen sulfate is used at a concentration of 3 to 5 (w/w) % at a temperature of 25 to 35° C. for 5 to 10 minutes to wash the surface of the pineapple.

Then, the washed pineapple may be crushed.

Specifically, the pineapple may be crushed to a diameter of 0.5 to 2 cm by means of a crusher or a mixer so that the active ingredients of the pineapple can be extracted within a short time.

Thereafter, the pineapple may be steamed.

Steaming of the pineapple means heating and steaming the crushed pineapple with water vapor. In the present invention, in order to effectively steam the pineapple, two steaming steps, that is, a first steaming step and a second steaming step, may also be performed.

For example, the first steaming step in the process of steaming the pineapple is a step of heating the crushed pineapple with water vapor at a temperature of 60 to 70° C. for 10 to 30 minutes, and the second steaming step is a step of heating the heated pineapple with water vapor at a temperature of 80 to 90° C. for 1 to 3 hours.

In the present invention, if the first steaming step is performed at a temperature lower than 60° C. or for a shorter than 10 minutes, a problem may arise in that it is difficult to uniformly steam the surface and outer portion of the pineapple, and if the first steaming step is performed at a temperature higher than 70° C. or for longer than 30 minutes, the surface and inner portion of the pineapple may be preheated, and thus no further increase in the effect can appear.

If the second steaming step is performed at a temperature lower than 80° C. or for shorter than 1 hour, a problem may arise in that the pineapple is not sufficiently steamed, making it difficult to prepare a pineapple extract within a short time in a subsequent process, and if the second steaming step is performed at a temperature higher than 90° C. or for longer than 3 hours, a problem may arise in that the active ingredients of the pineapple are volatilized or the physical properties of the pineapple are weakened.

Next, the steamed pineapple may be dried.

Drying of the steamed pineapple may be performed at a temperature of 60 to 70° C. for 30 to 150 minutes. If drying of the pineapple is performed at a temperature lower than 60° C. or for shorter than 30 minutes, a problem may arise in that it is difficult to sufficiently remove water remaining in the steamed pineapple, and if the second steaming step is performed at a temperature higher than 70° C. or for longer than 150 minutes, a problem may arise in that the remaining water is no longer removed or the physical properties of the pineapple change.

Next, the dried pineapple may be extracted, thereby preparing a pineapple extract.

Extraction of the dried pineapple may be performed using a known extraction method, such as a solvent extraction method, a hot-water extraction method or a supercritical extraction method. For example, the pineapple extract may be prepared by a method including the steps of: (a) extracting 1000 to 1200 g of the dried pineapple with 10 to 12 L of 70% (v/v) ethanol aqueous solution at 85 to 90° C. for 3 to 4 hours, followed by extraction with 4 to 6 L of 70% (v/v) ethanol aqueous solution at 85 to 90° C. for 2 to 3 hours, and concentrating the extract to a volume of 1000 to 1200 ml in a vacuum rotary evaporator; (b) diluting the extract to a 2- to 3-fold weight with water, centrifuging the dilution at 14,000 to 15,000rpm for 20 to 30 minutes to remove polymer cellulose and solids, and extracting the collected supernatant twice with a 1.2- to 1.5-fold weight of chloroform; and (c) removing the chloroform layer, and extracting the aqueous layer three times with a 1.2- to 1.5-fold weight of ethyl acetate, thereby preparing a pineapple extract.

The Dendropanax morbifera Lev extract that is used in the present invention is an extract from Dendropanax morbifera Lev. The Dendropanax morbifera Lev is an evergreen broad-leaved tree belonging to the genus Eleutherococcus of the family Araliaceae, and is a Korean native tree growing to a height of 15 m or higher. The young branches thereof are green and lustrous, the flowers bloom in June, and the fruits having a length of 7 to 19 mm ripen black. It is a warm temperate plant that grows in areas with a minimum temperature of −2° C. or above and an average annual temperature of 12 to 15° C. or above.

Globally, Dendropanax morbifera Lev is a rare species that grows only in the southwestern coast and island areas of the Korean Peninsula and in Jeju Island, and at present, the quantity thereof is extremely limited. The Dendropanax morbifera Lev is golden in color, and the golden color of the Dendropanax morbifera Lev becomes brighter when exposed to sunlight. Dendropanax morbifera Lev is characterized by forming a golden coating layer while the pattern and color of the product, unlike lacquer which gives a black color.

The Dendropanax morbifera Lev is generally composed of 66.7% non-volatile component, which is a coating component forming a golden coating layer, 10.8% aromatic component, 8.1% water, and 14.4% solid. In particular, the aromatic component is known to be composed of sesquiterpenes, including β-cubebene, γ-selinene and δ-cadinene. The aromatic component contained in the Dendropanax morbifera Lev is valuable as a benzoin that makes the mind and body clean and comfortable, and also has various pharmacological actions. Thus, it is highly useful as perfume, cosmetics, or functional foods and beverages.

The Dendropanax morbifera Lev extract that is used in the present invention may be prepared by the following method.

To prepare the Dendropanax morbifera Lev extract according to the present invention, Dendropanax morbifera stem is prepared, and then ground using a known grinding device such as a ball milling device, thereby preparing Dendropanax morbifera stem powder. The Dendropanax morbifera stem may be ground into powder having a particle diameter of 100 to 1000 μm and a specific surface area of 10 to 1000 m²/g.

If the Dendropanax morbifera stem is ground into powder having a particle diameter of less than 100 μm or a specific surface area of 10 m²/g, problems may arise in that the grinding time increases and workability is reduced by fine powder, and if the Dendropanax morbifera stem is ground into powder having a particle diameter of more than 1000 μm or a specific surface area of 1000 m²/g, a problem may arise in that the active ingredients contained in the Dendropanax morbifera stem powder are not easily extracted.

Next, the Dendropanax morbifera stem powder is dried to remove water. Drying of the Dendropanax morbifera stem powder may be performed in a dryer at a temperature of 25 to 30° C. and a humidity of 30 to 40% for 5 to 15 hours. According to the present invention, when the Dendropanax morbifera stem powder is dried under the above-described conditions, water contained in the Dendropanax morbifera stem powder can be evaporated gradually and evenly, and thus the useful components of the Dendropanax morbifera stem powder can be more easily extracted. In addition, the cell tissue of the Dendropanax morbifera stem powder can be destroyed, so that the penetration of air into the Dendropanax morbifera stem powder can be facilitated, whereby the useful components of the Dendropanax morbifera stem powder can be extracted thickly.

Thereafter, the dried Dendropanax morbifera stem powder may be steamed with water vapor.

Steaming of the dried Dendropanax morbifera stem powder may be performed by heating the powder with water vapor at a certain temperature under a certain pressure. For example, steaming of the dried Dendropanax morbifera stem powder may be performed by heating the powder with water vapor at a temperature of 145 to 155° C. and a pressure of 2 to 3 kgf/cm² for 1 to 5 hours.

In the present invention, if steaming of the dried Dendropanax morbifera stem powder is performed below the lower limits of the above-described process conditions, a problem may arise in that the Dendropanax morbifera stem powder is not sufficiently steamed, making it difficult to prepare a sufficient amount of an extract within a short time in a subsequent process, and if the steaming is performed above the upper limits of the above-described process conditions, a problem may arise in that the active ingredients of the Dendropanax morbifera stem powder are volatilized or a further increase in the steaming effect is insufficient.

Thereafter, the steamed Dendropanax morbifera stem powder is sonicated, and then the Dendropanax morbifera stem powder solid is removed, thereby preparing a Dendropanax morbifera stem powder extract.

To prepare the Dendropanax morbifera stem powder extract, the steamed Dendropanax morbifera stem powder may be mixed with a solvent to obtain a mixture, and the mixture may be placed in an ultrasonic extractor and sonicated, thereby preparing the Dendropanax morbifera stem powder extract. As the solvent, one or more solvents selected from among water or C1 to C4 alcohol may be used.

In addition, to prepare the Dendropanax morbifera stem powder extract, sonication of the mixture may be performed using a power of 200 to 300 watts at a vibration frequency of 50 to 100 KHz for 50 to 100 minutes.

Next, the Dendropanax morbifera stem powder extract may be centrifuged.

The Dendropanax morbifera stem powder extract contains the solvent and the useful components extracted from the Dendropanax morbifera Lev powder. When the Dendropanax morbifera stem powder extract containing the useful components extracted from the Dendropanax morbifera stem powder is centrifuged, it may be separated into a lower layer containing fine impurities of the Dendropanax morbifera stem powder, a middle layer located above the lower layer, and a supernatant.

Thereafter, the middle layer and supernatant of the centrifuged Dendropanax morbifera stem powder may be separated, and the separated middle layer and supernatant may be heated, thereby preparing a Dendropanax morbifera stem extract.

In the step of preparing the Dendropanax morbifera stem extract, the middle layer and supernatant of the centrifuged Dendropanax morbifera stem powder may be separated, and then heated at a temperature of 125 to 135° C. to remove the solvent, thereby preparing a gel-type Dendropanax morbifera stem extract containing the active ingredients extracted from the Dendropanax morbifera stem powder.

Thereafter, Dendropanax morbifera fresh leaf is prepared, and then washed to remove foreign matter attached to the Dendropanax morbifera fresh leaf.

At this time, washing of the Dendropanax morbifera fresh leaf may be performed using purified water containing sodium hydrogen carbonate dissolved therein. Preferably, the Dendropanax morbifera fresh leaf is washed in a solution containing 2 to 4(w/w) % sodium hydrogen carbonate at a temperature of 30 to 35° C. for 5 to 10 minutes.

Thereafter, the washed Dendropanax morbifera fresh leaf may be dried in a dryer. In the present invention, drying of the Dendropanax morbifera fresh leaf may be performed in dryer at a temperature of 20 to 25° C. and a humidity of 35 to 40% for 5 to 10 hours in order to prevent the chlorophyll of the Dendropanax morbifera fresh leaf from being destroyed in sunlight drying and the useful components thereof from being volatilized.

Next, the dried Dendropanax morbifera fresh leaf may be mixed with purified water, and then heated, thereby preparing a Dendropanax morbifera fresh leaf extract.

The Dendropanax morbifera fresh leaf extract may be prepared by 100 parts by weight of the Dendropanax morbifera fresh leaf with 1000 to 2000 parts by weight of purified water, and then heating the mixture at a temperature of 110 to 120° C. and a pressure of 2 to 4 kgf/cm² for 5 to 7 hours.

Next, the Dendropanax morbifera stem extract and the Dendropanax morbifera fresh leaf extract may be mixed with each other, thereby a Dendropanax morbifera LEV extract. Specifically, the Dendropanax morbifera LEV extract may be prepared by mixing the Dendropanax morbifera stem extract with the Dendropanax morbifera fresh leaf extract at a weight ratio of 3:7 to 7:3, and then storing the mixture at a temperature of 3 to 7° C. for 3 to 7 hours.

The 1,3-butylene glycol that is used in the present invention is a solvent that reduces viscosity and prevents the water of a high-purity atmosphere from forming a film.

The 1,3-butylene glycol can stabilize volatile substances, functions to prevent microbial spoilage, and has a very good distribution coefficient. Thus, it can be effectively used in the formulation of an antimicrobial composition.

The tourmaline that is used in the present invention has its own charge, and a weak permanent current (0.06 mA) flows therethrough. The radiant energy and far infrared rays emitted from tourmaline have the same wave length as a weak current (0.06 mA) essential for human life, and thus give cautery and stimulation effects to the weak part of humans, and restore the weak part to the original state. When the heat and water of tourmaline are adjusted, the tourmaline generates anions that change acidified skin and body fluids to weakly alkaline pH, thus making the skin healthy.

The far infrared rays and radiant energy from tourmaline have the effect of excreting skin waste and increasing the self-healing power of the skin, thereby facilitating metabolism in the human body.

The zeolite that is used in the present invention is a silicate mineral. The bond between atoms in the crystal structure is loosen, and thus when water filled therebetween is released with high heat, the framework of the zeolite can be maintained intact. Thus, other particulate substances can be adsorbed in the internal space of the zeolite. In particular, the zeolite has excellent deodorization and adsorption effects, and thus can adsorb and remove toxic components.

For example, the zeolite may have a specific surface area of 500 to 1000 m²/g and a particle diameter of 50 to 300 nm. To prepare the zeolite, sodium hydroxide is mixed with and dissolved in pure water, and sodium aluminate is added thereto, followed by stirring for 1 to 2 hours, thereby preparing a mixture. 1M Al₂O₃, 4M Na₂O, 10M SiO₂ and 125M H₂O are added to the mixture, and then stirred at a rotating speed of 250 to 300 rpm and a temperature of 30 to 35° C. for 3 to 5 hours. The stirred mixture is heated and stirred at a temperature of 85 to 105° C. and at a rotating speed of 150 to 200 rpm for 3 to 6 hours. The heated and stirred solution is crystallized using a microwave heat source at a temperature of 80 to 95° C. and at a rotating speed of 100 to 150 rpm for 1 to 3 hours, thereby synthesizing a zeolite crystal. The zeolite crystal is washed with pure water to remove unreacted material, and then heated and stirred in a dryer at a temperature of 75 to 85° C. for 3 to 5 hours. Next, the stirred zeolite crystal is adjusted to a pH of 6.5 to 7.5 with 3% nitric acid, and then an ammonium chloride (NH₄Cl ) solution was added thereto. Next, the mixture is stirred at a rotating speed of 100 to 150 rpm and a temperature of 30 to 40° C. for 15 to 20 hours, thereby preparing a zeolite crystallization solution. Then, 1000 to 1500 parts by weight of pure water is added to 100 parts by weight of the zeolite crystallization solution and stirred, after which 10 to 15 parts by weight of 12% silver nitrate (AgNO₃) solution is added thereto. Next, the resulting mixture is allowed to react at a temperature of 35 to 45° C. for 5 to 10 hours and washed. After the washing, the mixture is calcined in a dryer at a temperature of 550 to 600° C. for 10 to 12 hours, thereby preparing powder-type zeolite.

The photocatalyst is a substance that promotes a chemical reaction by receiving light. When energy higher than band-gap energy is irradiated to this photocatalyst, the photocatalyst generates electrons and holes, and a hydroxyl radical (—OH) is produced by the generated holes. Through the oxidative power of the hydroxyl radical, the gaseous or liquid organic material adsorbed on the surface is decomposed. This decomposition reaction is called ‘photooxidative reaction’.

The photocatalyst that is used in the present invention exhibits catalytic activity by absorbing light energy. Through the strong oxidative power generated by absorbing light energy, the photocatalyst oxidizes and decomposes toxic substances released from the sweat, waste or secretion of the human body. Due to this property, the photocatalyst is mainly used to purify organic wastewater and turbid water by decomposing bacteria, microorganisms, organic substances and the like.

The photocatalyst that is used in the present invention may be a powdery mixture of titanium dioxide (TiO₂) and silver nanopowder. When the titanium dioxide receives light, it decomposes toxic substances, such as benzene, toluene and formaldehyde, into water and carbon dioxide. In addition, the titanium dioxide has various advantages in that it is inexpensive and harmless to the human body. The silver (Ag) nanopowder is mixed with titanium dioxide and used to enhance the effect of the antibacterial photocatalyst. Addition of the silver (Ag) nanopowder can promote a photooxidative reaction, improve mixing and application properties, and increase antibacterial and antifungal activities.

The activated carbon that is used in the present invention is a carbon aggregate having fine pores developed therein. The internal surface area thereof can be changed by an activation process, and the activated carbon have excellent abilities to absorb and adsorb the surrounding liquid or gas by the internal carbon atom functional groups thereof.

In addition, in the present invention, the activated carbon may be used in powder form. This activated carbon in powder form can have increased adsorption ability compared to porous activated carbon. As the particle size of the activated carbon decreases, the portion between the particles is denser, and thus the adhesion between the particles increases, and these particles can be prevented from oxidation and can be hydrophobic.

In the present invention, the activated carbon powder has a strong ability to exterminate toxins in the body, and thus has excellent efficacy against various adult diseases, dermatitis and allergic symptoms, which are caused by the accumulation of toxicity in the body. The activated carbon powder may be prepared by a method including the steps of: cutting each of bamboo, oak and pine to a length of 500 to 1000 mm; carbonizing each of the cut cutting bamboo, oak and pine at a temperature of 1000 to 1500° C.; sorting and separating each of the carbonized activated carbons; and grinding each of the separated activated carbons to a particle diameter of 50 to 250 mesh.

In addition, the activated carbon that is used in the present invention may have a mass fraction of 90% or more, a packing density of 0.47 to 0.54 g/ml, a specific surface area of 1,200 to 2,500 m²/g, a pore diameter of 4 Å to 11 Å, an iodine adsorption capacity of 1,000 mg/g or more, a pore volume of 0.503 ml/g or more, a pH of 10 to 11, a phenol adsorption capacity of 18 ml/g or more, and an M·B decolorization capacity of 160 ml/g or more.

The glucosamine (C₆H₁₃NO₅) that is used in the present invention is a kind of amino sugar. It is a major precursor of biochemical association of glycoprotein and glycolipid. It is a part of the structure of polysaccharides such as chitosan or chitin, which are the main constituents of the exoskeletons various arthropods, including crustaceans, or the cell walls of fungi. It is known that the glucosamine is a major constituent of cartilage and is useful for joint health.

The dietary sulfur MSM (methyl sulfonyl methane) that is used in the present invention is an oxidized metabolite of dimethylsulfoxide (DMSO) having a sulfur content of 34%, and is a bioavailable sulfur in natural organic form. The sulfur component has a mysterious detoxification action against the heavy metals, harmful substances, and chemical poisons, which are the biggest enemies in the health line of modern people, compared to any other elements. Thus, it is called the 21^{st} century elixir of life.

The sulfur is one of the basic materials that constitute life, like water, and is the fourth most abundant mineral in the human body after calcium, phosphorus and potassium. It is found in all cells of the body, is a component of important amino acids, cystine, cysteine and methionine, and is essential for the cell's own life.

The methyl sulfonyl methane has an osteoarthritis alleviating effect, a systemic scleroderma alleviating effect, an anti-inflammatory effect, a neuroblocking effect (analgesic effect), and a vasodilation effect.

The vanillyl butyl ether (VBE) that is used in the present invention has the function (or property/effect) of imparting a warming sensation to the skin through the nervous system.

Hereinafter, examples of the sports muscle care tape according to the present invention will be described in more detail with reference to the accompanying drawings.

### EXAMPLE 1

A sports muscle care tape was prepared by forming an adhesive layer on the bottom surface of a substrate tape. The adhesive layer included 100 parts by weight of an adhesive and 10 parts by weight of an adhesive composition.

At this time, the adhesive composition included 2 parts by weight of germanium, 10 parts by weight of the polymer expressed by formula 1, 4 parts by weight of sorbic acid, 2 parts by weight of a cucumber extract, 1 part by weight of a pineapple extract, 3 parts by weight of a Dendropanax morbifera Lev extract, 7 parts by weight of 1,3-butylene glycol, 2 parts by weight of tourmaline, 3 parts by weight of zeolite, 3 parts by weight of a photocatalyst, and 1 part by weight of activated carbon. In addition, the photocatalyst was a mixture of 2 parts by weight of titanium dioxide and 0.3 parts by weight of silver nanopowder.

### EXAMPLE 2

A sports muscle care tape was prepared using the same adhesive composition as used in Example 1. However, in Example 2, the adhesive layer further included, based on 100 parts by weight of the adhesive composition, 3 parts by weight of glucosamine, 5 parts by weight of methyl sulfonyl methane, and 8 parts by weight of vanillyl butyl ether.

### Comparative Example

A muscle care tape including no adhesive composition was prepared and used as a sports muscle care tape according to a Comparative Example.

### 1. Deodorization Test

A deodorization test for the sports muscle care tape and the sports muscle care tape according to the Comparative Example was performed, and the results of the test are shown in Table 1 below.

TABLE 1

| | | Concentration (ppm) | |
|---|---|---|---|
| | Elapsed | of Comparative | Concentration (ppm) |
| Test items | time (min) | Example | of Example 1 |
| Deodorization | 30 | 640 | 388 |
| test | 60 | 632 | 345 |
| | 150 | 625 | 300 |
| | 300 | 605 | 255 |

Referring to Table 1 above which showing the deodorization test results obtained by measuring the concentration of ammonia test gas using FTIR, it can be seen that the deodorization rate of the sports muscle care tape prepared according to Example 1 increased with the passage of time.

### 2. Antibacterial Test

An antibacterial test for the sports muscle care tape prepared according to Example 1 was performed using Escherichia coli ATCC 25922 and Pseudomonas aeruginosa ATCC 15442, and the results of the test are shown in Table 2 below.

TABLE 2

| | | | Concentration |
|---|---|---|---|
| | | Initial | (CFU/40 p) |
| Test items | Samples | concentration | after 48 hours |
| Test using | Comparative | 550 | 955 |
| Escherichia coli | Example | | |
| | Example 1 | 550 | 260 |
| Test using | Comparative | 420 | 750 |
| Pseudomonas | Example | | |
| aeruginosa | Example 1 | 420 | 295 |

Referring to Table 2 above, it could be seen that the bacterial concentration in the sports muscle care tape according to the Comparative Example increased with the passage of time, whereas the bacterial concentration in the sports muscle care tape according to Example 1 decreased with the passage of time.

In the unit of the concentration, CFU denotes colony forming units, and 40 p denotes 0.04 mL.

### 3. Measurement of Capillary Blood Flow

The sports muscle care tape prepared according to the Example was attached to the skin of a test subject, and the capillary blood flow in the test subject was measured before and after attachment of the sports muscle care tape.

FIG. 2a is a photograph showing capillary blood flow before attachment of the sports muscle care tape prepared according to the present invention, and FIG. 2b is a photograph showing capillary blood flow after attachment of the sports muscle care tape prepared according to the present invention.

Referring to FIGS. 2a and 2b, it can be seen that capillary blood flow after attachment of the sports muscle care tape prepared according to the present invention was active. This suggests that the sports muscle care tape prepared using materials that emit large amounts of far infrared rays and anions can promote blood circulation and metabolism, thereby alleviating fatigue.

### 4. Measurement of Heat Generation Effect

The difference in the temperature of a body portion between before and after (after 3 minutes) attachment of the muscular care tape prepared according to Example 1 was measured.

TABLE 3

| Remarks | Before attachment | After attachment |
|---|---|---|
| Skin temperature (° C.) | 37.5° C. | 40.9° C. |

Referring to Table 3 above, it could be seen that before the muscle care tape prepared according to Example 2 was attached to the skin muscle of the affected part after exercise, the temperature of the skin muscle was 37.5° C., and at 3 minutes after attachment of the tape, the temperature of the skin muscle was 40.9° C.

Referring to Table 3 above, it could be seen that the muscle care tape prepared according to Example 2, which includes glucosamine, methyl sulfonyl methane and vanillyl butyl ether in the adhesive layer, promoted heat generation, thereby promoting blood circulation and metabolism in the taped body area.

As described above, the sports muscle care tape prepared according to the present invention can perform the basic functions of a sports tape and, at the same time, effectively protect an affected part by absorbing external impact.

In addition, the sports muscle care tape prepared according to the present invention can promote blood circulation and metabolism, has antibacterial activity, can deodorize the sweat, odor and the like generated in taped skin, and thus can be hygienically used.

It will be sufficiently understood that various embodiments according to the technical idea of the present invention can provide various effects not stated in detail.

While the preferred embodiments of the present invention have been described with reference to the accompanying drawings, it will be understood by those skilled in the art to which the present invention pertains that the present invention may be embodied in other specific forms without departing from the technical spirit or essential characteristics of the present invention. Therefore, the embodiments described above are considered to be illustrative in all respects and not restrictive.

What is claimed is:

1. A sports muscle care tape comprising: a substrate tape extending in a lengthwise direction; and an adhesive layer formed on a bottom surface of the substrate tape,
wherein the adhesive layer comprises 100 parts by weight of an adhesive and 5 to 15 parts by weight of an adhesive composition,
wherein the adhesive composition comprises germanium, a polymer represented by the following formula 1, sorbic acid, a cucumber extract, a pineapple extract, a Dendropanax morbifera Lev extract, 1,3-butylene glycol, tourmaline, zeolite, a photocatalyst, and activated carbon: 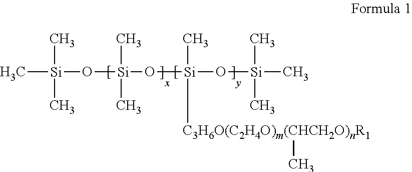
wherein x is an integer ranging from 500 to 30,000; y is an integer ranging from 100 to 10,000; m and n are each independently an integer ranging from 10 to 10,000; and R₁ is hydrogen or an alkyl group having 2 to 5 carbon atoms.

2. The sports muscle care tape of claim 1, wherein the adhesive composition comprises 1 to 3 parts by weight of germanium, 5 to 15 parts by weight of the polymer represented by formula 1, 3 to 5 parts by weight of sorbic acid, 1 to 3 parts by weight of the cucumber extract, 0.5 to 1.5 parts by weight of the pineapple extract, 2 to 4 parts by weight of the Dendropanax morbifera Lev extract, 5 to 10 parts by weight of 1,3-butylene glycol, 1 to 3 parts by weight of tourmaline, 2 to 4 parts by weight of zeolite, 2 to 4 parts by weight of the photocatalyst, and 0.5 to 1.5 parts by weight of activated carbon, and the photocatalyst is composed of a mixture of 1 to 3 parts of titanium dioxide and 0.1 to 0.5 parts by weight of silver nanopowder.
